(19) United States
(12) Patent Application Publication
(10) Pub. No.: US 2022/0333803 A1
(43) Pub. Date: Oct. 20, 2022

(54) Device for the Treatment of Air Comprising an Ionisation Module

(71) Applicant: Ionair AG, Luzern (CH)

(72) Inventor: Beda WEIBEL, Luzern (CH)

(73) Assignee: Ionair AG, Luzern (CH)

(21) Appl. No.: 17/640,218

(22) PCT Filed: Sep. 4, 2020
(86) PCT No.: PCT/EP2020/074730

§ 371 Date: Mar. 3, 2022

(30) Foreign Application Priority Data
Sep. 5, 2019 (DE) 10 2019 123 885.4

(51) Int. Cl. F24F 8/30; F24F 11/72

(52) U.S. Cl. CPC F24F 8/30; F24F 11/72; F24F 2110/20

(57) ABSTRACT

The invention relates to devices for the treatment of air of at least one room. These are distinguished in particular by the fact that air which is low in bacteria, germs and odours can be provided in at least one room.

For this purpose, the device has at least one supply air line which supplies treated air and in each case has at least one air treatment device having at least one ionization module, an air flow sensor and an air humidity sensor. At least one exhaust air line has at least one air quality sensor. Furthermore, at least one dust-measuring device arranged in front of the ionization module and/or at least one dust-measuring device arranged in the exhaust-air line are present. In addition, the ionization module, the sensors and the dust measuring devices are connected to a data processing system for increasing or decreasing the ionization intensity. Furthermore, at least one device measuring the load current of the ionization module is connected to the data processing system, which assigns an operating state of the ionization module in the event of a load current change as a function of the measured values of the dust measuring device.

The invention relates to devices for the treatment of air of at least one room.

The air in a room can be treated with devices for ionization. Bacteria and other germs are killed and large molecules are split into small-molecular fragments. Complex and large molecules are, among other things, odorous substances, and thus odour pollution can be suppressed with an air ionization. Furthermore, micro-organisms in the air can also be effectively reduced.

In devices for ionization, electric fields between two electrodes with voltage potentials are utilized in order to generate ions by impact ionizations by means of gas discharges. Known ionization tubes in the form of glass tubes with an inner electrode and an outer electrode can be used for this purpose. If a sufficiently high electric voltage is applied for the gas discharge, the glass of the wall forms a dielectric in which a large electric field is present. The air flowing through is enriched with ions.

DE 43 34 956 C2 discloses a process for the treatment of air with ions and a device for carrying out the process, the long-term stability of an ionization apparatus being increased. The main focus is on avoiding increased ozone production. For this purpose, sensors in the form of an air quality sensor, an air flow sensor and an air humidity sensor are used. When both external sources of interference occur, such as, for example, in the case of smog, an inversion weather situation, thunderstorms, external energy fields and internal sources of interference as a result of the operation of electrical appliances, the load on ozone in the supply air can increase to an undesirable extent and lead to a limit value being exceeded.

DE 100 07 523 C2 describes a method for treating air with ions and a device for carrying out the method, in which an ozone sensor is additionally used to determine the ozone content.

The invention stated in claim 1 is based on the task of providing air that is low in bacteria, germs and odours in at least one room.

This task is achieved by the features recited in patent claim 1.

The devices for the treatment of air of at least one room are distinguished in particular by the fact that air which is low in bacteria, germs and odours can be provided in at least one room.

Within the meaning of the invention, the air treatment device has at least one supply air line which supplies treated air and in each case has at least one air treatment device having at least one ionization module with a plurality of ionization tubes, an air flow sensor and an air humidity sensor. According to the invention, at least one exhaust air line has at least one air quality sensor. According to the invention, at least one dust-measuring device arranged upstream of the ionization module in the direction of flow of the supply air and/or at least one dust measuring device arranged in the exhaust air line are further provided. According to the invention, a data processing system is connected to the ionization module, the air flow sensor, the air humidity sensor, the air quality sensor and the dust measuring device upstream of the ionization module and/or the dust measuring device of the exhaust air line in order to increase or reduce the ionization intensity in accordance with the measured values of the air flow sensor, the air humidity sensor, the air quality sensor and the dust measuring device upstream of the ionization module and/or the dust measuring device of the exhaust air line. Within the meaning of the invention, at least one device measuring the load current of the ionization module and/or at least one ionization tube of the ionization module is or are furthermore connected to the data processing system, the data processing system assigning an operating state of the ionization module in the event of a change in the load current of the ionization module or at least one ionization tube and/or in the event of a change in the load current of the ionization module or at least one ionization tube as a function of the measured values of the dust-measuring device.

With the aid of the device, an improved room air for the room user is achieved. This is done taking into account the parameters that influence the ionization effect and taking into account applicable limit values. For this purpose, outside air and/or circulating air can enter the room as exhaust air of the room via the air treatment device after the treatment of air as supply air via the supply air line. As a result, the room user enjoys reduced VOC concentrations, reduced bacteria and germ count, as well as reduced PM concentrations (PM—Particulate Matter, equal to fine dust) in the breathed room air. VOCs in this context are volatile organic compounds (VOCs), for example tobacco smoke, kitchen fumes, vapours from humans or outgassing of materials such as, for example, furniture, carpets, adhesives and the like. With the device, the indoor air hygiene is thus increased.

For this purpose, the air quality sensor used can be a metal oxide semiconductor gas sensor which can also be used to detect volatile organic substances/compounds (VOCs).

By means of the at least one device measuring the load current of the ionization module and/or at least one ionization tube of the ionization module, an ionization power of at least one ionization tube which decreases as a result of ageing can advantageously be determined as the operating state of the ionization module. For this purpose, the load current is the electric current flowing through the ionization module and/or the at least one ionization tube during operation. In conjunction with the dust measuring device, a decreasing ionization power can be determined when at least one ionization tube is contaminated as the operating state of the ionization module. For this purpose, a change in load current compared to a nominal load current is determined. The nominal load current can be the load current during initial commissioning or restarting (e.g. after maintenance) of the air treatment device.

In embodiments, the air treatment device is designed as a structural unit of an air conditioning system. In addition, the air treatment device can also be a structural unit of a room air device.

For this purpose, the operating state can be assigned and displayed as at least one characteristic value in a graphic and/or a table and/or a colour scale. Advantageously, the respective time is allocated to this.

Advantageous embodiments of the invention are specified in claims 2 to 11.

According to one embodiment of the subject matter of the invention, the device measuring the load current of an ionization tube of the ionization module is connected to the data processing system, which is a data processing system which assigns this change to the operating state of the ionization tube in the event of a change in the load current of the ionization tube and/or in the event of a change in the load current of the ionization tube as a function of the measured values of the dust measuring device upstream of the ionization module and/or of the dust measuring device of the exhaust air line.

According to a further embodiment of the subject matter of the invention, the exhaust air line of the room is connected to a line for discharging used air and/or to a line for supplying outside air. Furthermore, the line supplying outside air is connected to the air treatment device.

According to a preferred embodiment, the used air discharging line has a first fitting and the line supplying the outside air has a second fitting. The actuating devices of the fittings are connected to the data processing system for changing the respective air flow. Thus, among other things, outside air can be supplied or a recirculating air operation can be set. In embodiments, the fittings are designed as valves or flaps.

According to a further preferred embodiment, the line supplying outside air has a further dust measuring device for measuring the dust of the outside air. The dust measuring device in the line supplying outside air is connected to the data processing system, which increases the ionization power of the ionization module and/or the air flow as a function of the value of the dust measuring device in the line supplying outside air when dust occurs or is increased in the outside air.

According to a further embodiment of the invention, the air flow sensor, the air humidity sensor, the air quality sensor and the dust measuring device upstream of the ionization module and/or the dust measuring device of the exhaust air line are connected to the data processing system, which determines a room air concentration as a function of the room size and of measured values of the air flow sensor, the air humidity sensor, the air quality sensor and the dust measuring device upstream of the ionization module and/or the dust measuring device of the exhaust air line as a model with weights of the air flow, the air humidity, the air quality and the dust.

According to a further embodiment of the invention, at least one ozone sensor is or are arranged in the supply air line and/or in the exhaust air line and/or in the space and/or in the line supplying outside air. The ozone sensor or the ozone sensors is or are connected to the data processing system which determines the ionization intensity from the measured values of the air humidity sensor, the air flow sensor, the air quality sensor and the dust measuring device upstream of the ionization module and/or the dust measuring device of the exhaust air line and the ozone sensor or the ozone sensors in accordance with the weights of the air humidity, the air flow, the air quality and the dust and an ozone limit value and, in the event of a change in the ionization intensity, changes the ionization power in such a way that compensation and, in accordance with the change, a further reduction or increase in the ionization intensity takes place without an impairing or damaging ozone limit value being exceeded.

According to a further embodiment of the subject matter of the invention, at least one carbon dioxide sensor is or are arranged in the supply air line and/or in the exhaust air line and/or in the space, which sensor or sensors is or are furthermore connected to the data processing system. The data processing system is a data processing system which changes the supply air and/or the air flow in the event of a change in the carbon dioxide, taking into account the ionization intensity, in such a way that the ionization intensity is determined as a function of the measured values of the air humidity sensor, the air flow sensor, the air quality sensor and the dust measuring device upstream of the ionization module and/or the dust measuring device of the exhaust air line, and the ionization intensity is reduced or increased in the event of a change, and a carbon dioxide limit value is maintained. This takes place with the supply of outside air, for which purpose fittings can be provided.

According to a further embodiment of the subject matter of the invention, the ionization module having ionization tubes is connected to the data processing system which controls the ionization power of the ionization tubes with pulse width modulation (PWM) by switching periods smaller than or greater than 100 periods from the power supply system. In preferred embodiments, these can be periods greater than 0 and less than or equal to 100 periods. In further embodiments, the periods may be greater than 0 and less than or equal to 200 periods. When the load current of at least one ionisation tube changes, the pulse width modulation is changed according to the load current change.

According to a further embodiment of the invention, the device measuring the load current of an ionization tube of the ionization module and/or the device measuring the load current of the ionization tubes of the ionization module is or are connected to the data processing system in such a way that, in the event of a change in the load current of at least one ionization tube, the load currents of the other ionization tubes are changed with respect to a known nominal load current.

According to a further embodiment of the invention, the data processing system is connected to a transmitter of electromagnetic waves and/or a transceiver and/or to a data network and/or a computer network. The measured values of the air flow sensor, the air humidity sensor, the air quality sensor and the dust measuring device can thus be interrogated remotely.

For the realization of the invention, it is also expedient to combine the above-described embodiments, embodiments and features of the claims in each arrangement.

Brief description OF the DRAWINGS The invention will be better understood and further objects and advantages thereof will become more apparent from the ensuing detailed description of preferred embodiments taken in conjunction with the drawings.

What is shown is:

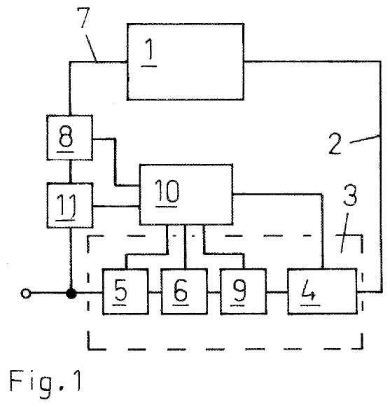

FIG. 1 shows a device for the treatment of air of at least one room and

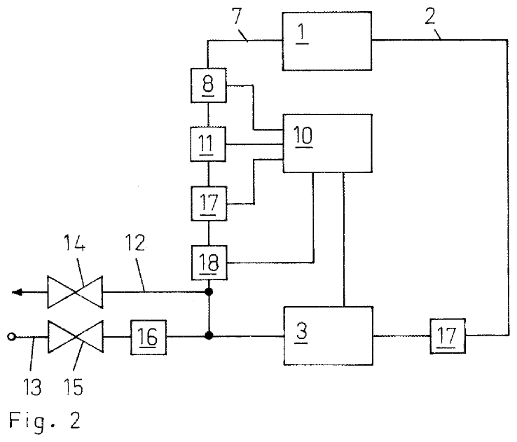

FIG. 2 shows a device for the treatment of air of at least one room with additional ozone sensors, an additional carbon dioxide sensor and a further dust measuring device.

The invention relates to a air treatment device of at least one room (1). Said device consists essentially of an air supply line (2) which supplies treated air and comprises a air treatment device (3) comprising an ionisation module (4) with several ionisation tubes, an air flow sensor (5) and an air humidity sensor (6), an exhaust air line (7) with an air quality sensor (8), a dust measuring device (9) which is arranged in front of the ionisation module (4) and at least one dust measuring device (11) which is arranged in the exhaust air line (7), a data processing system (10) which is connected to the ionisation module (4), the air flow sensor (5), the air humidity sensor (6), the air quality sensor (8) and the dust measuring device (9) in front of the ionisation module (4) and the dust measuring device (11) of the exhaust air line (7).

FIG. 1 shows a device for the treatment of air of at least one room 1 in a schematic representation.

The dust measuring device 9 is designed in such a way that, in particular, fine dust can also be detected. Devices for dust measurement and fine dust measurement are known.

Outside air and/or circulating air as exhaust air of the room 1 passes via the device for air conditioning 3 as supply air via the supply air line 2 into the room 1. The air treatment device 3 has the ionisation module 4 with several ionisation tubes, the air flow sensor 5, the air humidity sensor 6 and the dust measuring device 9 in front of the ionisation module 4. The exhaust air line 7 for removing room air has the air quality sensor 8 and the dust measuring device 11 of the exhaust air line 7. The air quality sensor 8 can in particular be a metal oxide semiconductor gas sensor, also for volatile organic substances/compounds VOC. The exhaust air line 7 thus simultaneously represents a circulating air line, exhaust air being supplied again as supply air. For this purpose, the latter is connected to the device 3 for air treatment. The ionization module 4, the air flow sensor 5, the air humidity sensor 6, the air quality sensor 8 and the dust measuring devices 9, 11, are connected to the data processing system 10. A device designed in this way for the treatment of air is a structural unit of a ventilation installation.

The data processing system 10 is designed to decrease or increase the ionisation intensity according to the measured values of the air flow sensor 5, the air humidity sensor 6, the air quality sensor 8 and the dust measuring devices 9, 11, so that the ionisation intensity and/or the air flow increases when the air humidity and/or the dust increases and when the air quality decreases.

The air flow sensor 5, the air humidity sensor 6, the air quality sensor 8 and the dust measuring devices 9, 11 are connected to the data processing system 10 for this purpose, which determines a room air concentration as a model with weights of the air flow, the air humidity, the air quality and the dust as a function of the room size and of measured values of the air flow sensor 5, the air humidity sensor 6, the air quality sensor 8 and the dust measuring device 9, 11. Furthermore, the data processing system 10 is a data processing system 10 determining the average room air concentration from the model of the room air concentration and assigning it to a limit value of the ionisation intensity.

FIG. 2 shows a device for the treatment of air of at least one room 1 with ozone sensors 17 which are additional to the device shown in FIG. 1, a carbon dioxide sensor 18 and a further dust measuring device 16 in a schematic representation. Reference is made below to the reference numerals of the device shown in FIG. 1.

In one embodiment, the device for air treatment of at least one room 1 may further comprise ozone sensors 17, a carbon dioxide sensor 18 and a further dust measuring device 16. One ozone sensor 17 each is located in the supply air line 2 and the exhaust air line 7. In addition, an ozone sensor 17 can also be arranged in a line 13 supplying outside air. The carbon dioxide sensor 18 is arranged in the exhaust air line 7 as a circulating air line. The further dust measuring device 16 is located in the line 13 supplying outside air. Thus, the exhaust air line 7 of the room 1 can also be connected to the device for air conditioning 3 as a circulating air line. The exhaust air line 7 is also connected to a used air discharge line 12 in the form of an exhaust air line with a first fitting 14 and a second fitting 15 with a line 13 supplying outside air. The actuating devices of the fittings 14, 15 are connected to the data processing system 10 for changing the respective air flow. For this purpose, the fittings 14, 15 can be flaps, for example.

The ozone sensors 17 are connected to the data processing system 10, which determines the ionization intensity from the measured values of the air flow sensor 5, the air humidity sensor 6, the air quality sensor 8, the dust measuring devices 9, 11, 16 and the ozone sensors 17 in accordance with the weights of the air flow, the air humidity, the air quality, the dust and the ozone. In the event of a change in the ionization intensity, the ionization power is changed by means of the data processing system 10 such a way that compensation and, corresponding to the change, a further reduction or increase in the ionization intensity takes place without an impairing or damaging ozone limit value being exceeded.

Especially ozone sensors 17 can be arranged in the supply air duct 2, in the exhaust air duct 7 for measuring the ozone in the room air and in the duct 13 supplying outside air for measuring the ozone of the outside air. If the ozone in the outside air is too high, the device for the treatment of air of at least one room can thus be operated in circulating air mode.

The carbon dioxide sensor 18 is connected to the data processing system 10 in such a way that, in the event of a change in the carbon dioxide, the supply air and/or the air flow is changed, taking into account the ionisation intensity, in such a way that, depending on the measured values of the air flow sensor 5, the air humidity sensor 6, the air quality sensor 8 and the dust measuring devices 9, 11, 16, the ionisation intensity is determined and, in the event of a change, a reduction or increase in the ionisation intensity is effected. A carbon dioxide limit value is maintained.

Further carbon dioxide sensors 18 connected to the data processing system 10 can be located in the supply air line 2 and/or in the space 1.

The ionization module 4 of the exemplary embodiment has a plurality of ionization tubes. At least one device measuring the load current of an ionisation tube in each case and/or a device measuring the load current of the ionisation tubes is or are connected to the data processing system 10, the data processing system 10 assigning an operating state of the ionisation module 4 in dependence on the measured values of the dust measuring device 9 upstream of the ionisation module 4 in the event of a load current change of the ionisation module or at least one ionisation tube and/or an operating state of the ionisation module 4 in the event of a load current change of the ionisation module or at least one ionisation tube.

The ionisation module 4 having ionisation tubes is also connected to the data processing system 10, which controls the ionisation power of the ionisation tubes with a pulse width modulation (PWM) by switching periods greater than 0 and less than or equal to 100 periods from the power supply. Furthermore, at least one device measuring the load current of an ionization tube of the ionization module 4 and/or a device measuring the load current of the ionization tubes can be connected to the data processing system 10, which changes the load currents of the other ionization tubes with respect to a known nominal load current in the event of a load current change of at least one ionization tube. When the load current of at least one ionisation tube changes, the pulse width modulation is changed according to the load current change.

### LIST OF REFERENCE SIGNS

1 room
2 Supply air line
3 Device for the treatment of air
4 Ionization module
5 Air flow sensor
6 Humidity sensor
7 Exhaust air line
8 Air quality sensor
9 Dust measuring device in front of the ionisation module 4
10 Data processing system
11 Dust measuring device in the exhaust air line 7
12 used air exhaust pipe
13 Outside air supply line
14 First valve
15 Second valve
16 Other dust measuring equipment
17 Ozone sensor
18 Carbon dioxide sensor

1. Device for the treatment of air of at least one room (1) with
at least one supply air line (2) supplying treated air, each having at least one device for air conditioning (3), each having at least one ionisation module (4) with a plurality of ionisation tubes, an air flow sensor (5) and an air humidity sensor (6),
at least one exhaust air line (7) with at least one air quality sensor (8),
at least one dust measuring device (9) arranged in front of the ionisation module (4) and/or at least one dust measuring device (11) arranged in the exhaust air line (7),
a data processing system (10) connected to the ionisation module (4), the air flow sensor (5), the humidity sensor (6), the air quality sensor (8) and the dust measuring device (9) upstream of the ionisation module (4) and/or the dust measuring device (11) of the exhaust air line (7) for increasing or decreasing the ionisation intensity in accordance with the measured values of the air flow sensor (5), the humidity sensor (6), the air quality sensor (8) and the dust measuring device (9) upstream of the ionisation module (4) and/or the dust measuring device (11) of the exhaust air line (7), and
at least one device measuring the load current of the ionisation module (4) and/or at least one ionisation tube of the ionisation module (4), which is or are connected to the data processing system (10),
wherein the data processing system (10) assigns an operating state of the ionisation module (4) in dependence on the measured values of the dust measuring device (9) in the event of a change in the load current of the ionisation module (4) or at least one ionisation tube and/or in the event of a change in the load current of the ionisation module (4) or at least one ionisation tube.

2. Device according to claim 1, characterized in that the device measuring the load current of an ionization tube of the ionization module (4) is connected to the data processing system (10), which is a data processing system (10) which assigns this change to the operating state of the ionization tube in the event of a change in the load current of the ionization tube and/or in the event of a change in the load current of the ionization tube as a function of the measured values of the dust-measuring device (9) upstream of the ionization module (4) and/or of the dust-measuring device (11) of the exhaust-air line (7).

3. Device according to claim 1 or 2, characterized in that the exhaust-air line (7) of the room (1) is connected to a line (12) which discharges used air and/or to a line (13) which supplies outside air, and in that the line (13) which supplies outside air is connected to the air treatment device (3).

4. Device according to claim 3, characterized in that the used-air-discharging line (12) has a first fitting (14) and the line (13) supplying the outside air has a second fitting (15), and in that the actuating devices of the fittings (14, 15) are connected to the data processing system (10) in order to change the respective air flow.

5. Device according to claim 3 or 4, characterized in that the line (13) supplying outside air has a further dust-measuring device (16) for measuring the dust of the outside air, and in that the dust-measuring device (16) in the line (13) supplying outside air is connected to the data processing system (10), which, in the event of the occurrence or increased occurrence of dust in the outside air, increases the ionization power of the ionization module and/or the air flow as a function of the value of the dust-measuring device (16) in the line (13) supplying outside air.

6. Device according to claim 5, characterized in that the air flow sensor (5), the air humidity sensor (6), the air quality sensor (8) and the dust measuring device (9) are connected upstream of the ionization module (4) and/or the dust measuring device (11) of the exhaust air line (7) to the data processing system (10), which determines a room air concentration as a function of the room size and of measured values of the air flow sensor (5), the air humidity sensor (6), the air quality sensor (8) and the dust measuring device (9) upstream of the ionization module (4) and/or the dust measuring device (11) of the exhaust air line (7) as a model with weights of the air flow, the air humidity, the air quality and the dust.

7. Device according to claim 6, characterized in that at least one ozone sensor (17) is or are arranged in the supply air line (2) and/or in the exhaust air line (7) and/or in the space (1) and/or in the line (13) supplying the outside air, and in that the ozone sensor (17) or the ozone sensors (17) is or are connected to the data processing system (10), which determines the ionization intensity from the measured values of the air humidity sensor (6), the air flow sensor (5), the air quality sensor (8) and the dust measuring device (9) upstream of the ionization module (4) and/or the dust measuring device (11) of the exhaust air line (7) and the ozone sensor (17) or the ozone sensors (17) in accordance with the weights of the air humidity, the air flow, the air quality and the dust and an ozone limit value, and changes the ionization power in the event of a change in the ionization intensity in such a way that compensation and, in accordance with the change, a further reduction or increase in the ionization intensity takes place without an adversely affecting or damaging ozone limit value being exceeded.

8. Device according to claim 7, characterized in that at least one carbon dioxide sensor (18) is or are arranged in the incoming air line (2) and/or in the outgoing air line (7) and/or in the space (1), and in that the carbon dioxide sensor (18) or the carbon dioxide sensors (18) is or are connected to the data processing system (10), which changes the incoming air and/or the air flow in the event of a change in the carbon dioxide, taking into account the ionization intensity, in such a way that the ionization intensity is determined as a function of the measured values of the air humidity sensor (6), the air flow sensor (5), the air quality sensor (8) and the dust-measuring device (9) upstream of the ionization module (4) and/or the dust-measuring device (11) of the outgoing air line (7), and a reduction or increase in the ionization intensity takes place in the event of a change, and a carbon dioxide limit value is maintained.

9. Device according to claim 8, characterized in that the ionization module (4) having ionization tubes is connected to the data processing system (10), which controls the ionization power of the ionization tubes with pulse width modulation (PWM) by means of switching periods from the mains.

10. Device according to claim 9, characterised in that the device measuring the load current of an ionisation tube of the ionisation module (4) in each case and/or the device measuring the load current of the ionisation tubes of the ionisation module (4) is or are connected to the data processing system (10) in such a way that in the event of a change in load current of at least one ionisation tube, the load currents of the other ionisation tubes are changed in relation to a known nominal load current.

11. Device according to claim 10, characterized in that the data processing system (10) is connected to a transmitter of electromagnetic waves and/or a transceiver and/or to a data network and/or a computer network.
